(19) United States
(12) Patent Application Publication
(10) Pub. No.: US 2023/0100744 A1
(43) Pub. Date: Mar. 30, 2023

(54) METHODS FOR ENRICHING MARROW INFILTRATING LYMPHOCYTES ("MILS"), COMPOSITIONS CONTAINING ENRICHED MILS, AND METHODS OF USING ENRICHED MILS

(71) Applicants: WINDMIL THERAPEUTICS, INC., Baltimore, MD (US); THE JOHNS HOPKINS UNIVERSITY, Baltimore, MD (US)

(72) Inventors: Ivan R. Borrello, Baltimore, MD (US); Kimberly A. Noonan, Baltimore, MD (US)

(73) Assignees: WINDMIL THERAPEUTICS, INC., Baltimore, MD (US); THE JOHNS HOPKINS UNIVERSITY, Baltimore, MD (US)

(21) Appl. No.: 17/802,352

(22) PCT Filed: Feb. 26, 2021
(86) PCT No.: PCT/US21/19841

§ 371 Date: Aug. 25, 2022

Related U.S. Application Data
(60) Provisional application No. 62/983,004, filed on Feb. 28, 2020.

(51) Int. Cl. A61K 35/17; C07K 14/575; C07K 14/705; C12N 5/0783; C07K 14/54

(52) U.S. Cl. CPC A61K 35/17; C07K 14/5759; C07K 14/70596; C12N 5/0636; C07K 14/54; C12N 2500/02

(57) ABSTRACT

A method for enriching or isolating tumor specific MILs is described. This method includes the steps of preparing MILs from the bone marrow of a cancer patient; evaluating the MILs for gene expression, metabolic profile, or phenotype; and selecting and isolating the MILs that exhibit the gene expression, metabolic profile, or phenotype. Compositions containing the MILs and methods of treating cancer with the enriched MILs are also described.

This application claims priority under 35 U.S.C. § 119 to U.S. Provisional Application 62/983,004, filed Feb. 28, 2020, the entirety of which is incorporated by reference herein.

### BACKGROUND OF THE INVENTION

### Field of the Invention

The present invention relates to methods and compositions for producing and using marrow infiltrating lymphocytes (MILs), and more specifically methods for producing MILs and compositions thereof that are enriched for the MILs.

### Brief Description of the Related Art

Marrow infiltrating lymphocytes (“MILs”) are the product of activating and expanding bone marrow T cells (see Noonan et al., Cancer Res. (2005) 65(5): 2026-2034). The bone marrow is a specialized niche in the immune system which is enriched for antigen experienced, central memory T cells. MILs that have been expanded under hypoxic conditions have been shown to confer immunologically measurable clinical benefits in patients with multiple myeloma (See U.S. Pat. No. 9,687,510). The bone marrow microenvironment has also been shown to harbor tumor-antigen specific T cells in patients with solid tumors such as breast, pancreatic and ovarian cancers (Schmitz-Winnenthal F. H. et al., Cancer Res. 2005 (November 1); 65(21):10079-1008). Therefore, development of tumor-specific MILs for use in cancer therapy, possibly in conjunction with traditional chemotherapy, represents an exciting development.

Hypoxic-activated MILs are a promising approach for adoptive cell therapy (ACT) due to their broader anti-tumor specificity and persistence. These characteristics stem from the intrinsic properties of the bone marrow (BM), known to be a reservoir for long-lived memory T cells and T-stem cell memory cells (TSCM), and expansion in hypoxia. Naïve and memory T cells are metabolically quiescent, favoring oxidative phosphorylation (OXPHOS) over glycolysis. In contrast, effector T cells favor glycolysis to fuel their rapid proliferation. More recent work has illuminated the roles that fatty acid (FA) uptake and oxidation (FAO) play in the maintenance of certain T-cell phenotypes, specifically tissue resident memory T-cells (TRM)—a subtype that, with TSCM, appears increasingly important in generating and maintaining the tumor specificity of MILs for extended periods of time. The underlying biology and mechanisms driving this phenotype of hypoxic-activated MILs to improve to ACT has not been previously described.

Hypoxic-activated MILs have now been characterized to determine their unique metabolic profile as well as unique gene expression profiles, which can be utilized to enrich and increase the usefulness of the MILs in adoptive T cell therapy.

### SUMMARY OF THE INVENTION

According to first aspect of the present invention, a method of enriching for tumor-specific MILs is provided, the method comprising preparing MILs from the bone marrow of a cancer patient; evaluating the MILs for gene expression, metabolic profile, or phenotype; selecting and isolating the MILs that exhibit the gene expression, metabolic profile, or phenotype; wherein said gene is selected from the group consisting of: IL-2, IL-7m IL015, IFNγ, IL-2Ra, CD69, CXCR6, CXCR4, CD127, TCF1, leptin, ghrelin, FABP5, CD36, and combinations thereof; wherein said metabolic profile is selected from the group consisting of upregulation of oxidative phosphorylation, upregulation of glycolytic machinery, fatty acid oxidation, and combinations thereof; and wherein said phenotype is selected from the group consisting of: a) an increase in mitochondrial proteins selected from the group consisting of TOMM20, CPT1a, SDHa, and combinations thereof, b) an increase in mTOR signaling, c) an increase in glycolytic machinery selected from the group consisting HK2, GLUT1, and combinations thereof, d) increased baseline oxygen consumption rate, e) spare respiratory capacity, f) extracellular acidification rate, and g) combinations thereof.

It is another aspect of the present invention to provide the method as described above, wherein said preparing comprises expanding the MILs under hypoxic conditions.

It is another aspect of the present invention to provide the method as described above, wherein said hypoxic conditions comprise incubating the MILs in an environment having about 0% oxygen to about 6% oxygen.

It is another aspect of the present invention to provide the method as described above, wherein said hypoxic conditions comprise incubating the MILs in an environment having less than 5% oxygen.

It is another aspect of the present invention to provide the method as described above, wherein said hypoxic conditions comprise incubating the MILs in an environment having between 0.5% and 1.5% oxygen.

It is another aspect of the present invention to provide the method as described above, wherein said preparing expanding the MILs under hypoxic conditions for about 1 to 12 days.

It is another aspect of the present invention to provide the method as described above, wherein said preparing expanding the MILs under hypoxic conditions for about 2 to 5 days.

It is another aspect of the present invention to provide the method as described above, wherein said preparing expanding the MILs under hypoxic conditions for about 3 to 4 days.

It is another aspect of the present invention to provide the method as described above, wherein said preparing comprises expanding the MILs under hypoxic conditions, followed by culturing the hypoxic-activated MILs in a normoxic environment.

It is another aspect of the present invention to provide the method as described above, wherein said normoxic environment comprises at least about 7% oxygen.

It is another aspect of the present invention to provide the method as described above, wherein said normoxic environment comprises about 7% to about 21% oxygen.

It is another aspect of the present invention to provide the method as described above, wherein said normoxic environment comprises about 21% oxygen.

It is another aspect of the present invention to provide a composition comprising tumor-specific MILs produced by the method as described above.

It is another aspect of the present invention to provide the composition as described above, wherein the tumor-specific MILs comprise characteristics as compared to PBLs or T cells grown under normoxic-only conditions, wherein the characteristics are selected from the group consisting of: 1) increased cytotoxicity, 2) persistence over time in vivo, 3) inducement of long-term memory, 4) expression of a beneficial cytokine profile for cytotoxicity, and 5) combinations thereof.

It is another aspect of the present invention to provide a method of treating a subject having cancer, said method comprising administering the composition as described above to the subject.

It is another aspect of the present invention to provide the method as described above, wherein the cancer is a hematological cancer.

It is another aspect of the present invention to provide the method as described above, wherein the hematological cancer is multiple myeloma.

It is another aspect of the present invention to provide the method as described above, wherein the cancer is a solid tumor.

It is another aspect of the present invention to provide a method of isolating tumor-specific MILs comprising incubating bone marrow aspirate from a patient having cancer in a hypoxic environment, followed by culturing in a normoxic environment, wherein the tumor specific MILs comprise characteristics as compared to PBLs or T cells grown under normoxic-only conditions, wherein the characteristics are selected from the group consisting of: 1) increased cytotoxicity, 2) persistence over time in vivo, 3) inducement of long-term memory, 4) expression of a beneficial cytokine profile for cytotoxicity, and 5) combinations thereof.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the compositions and methods, given only by way of example, and with reference to the accompanying drawings, in which:

FIGS. 1A, 1B, and 1C show in vivo fold expansion of MILs compared to PBLs under either normoxia or hypoxia conditions, including out to 180+ days.

FIGS. 2A, 2B, 2C, 2D, and 2E show that a unique population of CD8+, CD69+, and CD127+ cells was identified only in the bone marrow of patients receiving hypoxic aMILs, and normoxic aMILs or traditional transplant without aMILs failed to show this population. FIG. 2 shows that CD69+ cells within the hypoxia aMILs group are also Ki67 low and TCF1 high, suggesting they are hypo-proliferative and stem-like, while in normoxia, the inverse is true.

FIGS. 3A, 3B, 3C, 3D, 3E, and 3F show that RNA-sequencing reveals unique gene expression patterns in aMILs following initial hypoxic activation. Five paired samples (matched bloods and bone marrows from the same patients) were processed and activated in either normoxic or hypoxic conditions. Sequencing was run on isolated CD3+ T-cells. Genes of interest include KLF4, APOE, CD36, Rab32, and IL18 as well as many others involved in metabolism, activation, adhesion, and stemness.

FIGS. 4A, 4B, 4C, and 4D show that the glycolytic machinery is upregulated by MILs in hypoxia.

FIG. 4E shows that mTOR signaling is upregulated by MILs in hypoxia, representing a high baseline 02 consumption and increased expression of mitochondrial proteins as well as increased mTOR signaling.

FIGS. 4F and 4G show that glycolysis and glycolytic machinery are also upregulated in MILs activated in hypoxia. They have higher ECAR and increased uptake of glucose when compared to both normoxic aMILs and hypoxic aPBLs.

FIGS. 4H and 41 show that mitochondrial proteins are upregulated by MILs in hypoxia.

FIG. 5A shows the experimental workflow for the flow cytometry analysis and statistical analysis.

FIGS. 5B and 5C show that high-dimensional analysis of CD8+ T cells identifies BM-enriched subsets. tSNE analysis of concatenated CD8+ T cells from peripheral blood (PB) and bone marrow (BM) of newly diagnosed multiple myeloma (NDMM) patients is shown in FIG. 5B, and the frequency of the 18 Phenograph clusters in PB (blue) and BM (red) samples are shown in FIG. 5C.

FIGS. 6A, 6B, and 6C show that multiple myeloma BM T cells are enriched in CD69+ T cells. FIG. 6A shows a heat map showing the integrated MFI of specific markers in discrete Phenograph clusters identified in FIG. 5C. Clusters 15, 17, and 18 were significantly enriched in the bone marrow. FIG. 6B shows the median frequencies of clusters 15, 17, and 18 along with CD69+ and CD8+ T cells within peripheral blood and bone marrow samples. FIG. 6C shows representative dot plots showing manual gating with CD69+ CD8+ population in peripheral blood (left) and bone marrow (right) samples.

FIG. 7 shows that bone marrow CD69+ CD8+ T cells display an effector phenotype and a tissue resident-like (TRM) signature. BM TRM cells display a partial exhausted phenotype as exemplified by expression of PD1 and lack of TIM3, TIGIT, and 2B4. Higher expression of CXCR6 and CCR5 associated with lower Tbet and Ki67 levels suggests retained stem-like/proliferative capacity.

FIGS. 8A and 8B shows that CD127 (IL7R) identifies a subset of stem-like BM-resident CD8+ T cells. Phenograph clustering identified Cluster 15 as BM-enriched and CD127+, while both Cluster 17 and 18 lack CD127 expression. FIG. 8A shows that CD69+CD127+ CD8+ T cells display increased expression of the stemness marker TCF1, as well as lower PD1 and TIGIT levels. FIG. 8B shows that representative histograms depicting expression of TCF1 and PD1 in CD69+ CD127+ and CD69+ CD127−.

FIG. 9 shows that HLA-DR+ APCs in the bone marrow may form a supportive niche for BM-enriched CD8+ T cells and identification of APC subsets in PB and BM samples. BM APCs are characterized by a higher frequency of B cells and lower frequencies of both CD11c+ and CD11b+ HLA-DR+ APCs (three bottom/left panels). The ratio of CD11b−/CD11b+ HLA-DR+ APCs positively correlated with the frequency of CD8+ CD69+ T cells in the BM suggesting the existence of a BM-restricted niche that may support and differentiate stem-like CE8+ T cells thereby maintaining an effective anti-myeloma immune response (top/right panel).

FIG. 10 shows results when MILs were either grown in normoxia or hypoxia for 3 days and then converted to normoxia for a total of 10 days in culture. Then, RT-PCR was conducted for anti-apoptotic proteins of the cells either immediately after coming out of hypoxia or 7 days later. Greater expression of Bcl-2, Bcl_{XL}, as well as the hypoxia-inducible genes, VHL and NOS that was sustained at the late time point (see left panel). This also correlated with upregulation of a favorable cytokine profile as shown by greater expression of IL-2, IL-7, IL-15, IFNγ and IL-2Ra (see right panel).

FIG. 11 shows the metabolomic profile of MILs in hypoxia. A Seahorse analysis revealed a distinctive pattern of hypoxic MILs. Specifically, they had an increase in baseline 02 consumption, spare respiratory capacity, mitochondrial mass, mTOR signaling. This all translates into better tumor-specific cytotoxicity of autologous myeloma cells.

FIG. 12 shows that CD69+ tissue resident memory (Trm) play a significant role in mediating the local tissue protective immunity and immunosurveillance. They are enriched in the BM and play a key role in imparting long-term immunity. Shown here is BM from patients undergoing a stem cell transplant transplanted either without MILs or MILs grown in normoxia or hypoxia and obtained on d28 post-transplant. Expansion in hypoxia upregulates CD69 expression on MILs with co-expression of the IL7R (CD127)—a marker of long-term memory T cells.

### DETAILED DESCRIPTION OF EXEMPLARY EMBODIMENTS

Referring to the drawing figures, like reference numerals designate identical or corresponding elements throughout the several figures.

As used herein and unless otherwise indicated, the term “about” is intended to mean±5% of the value it modifies. Thus, “about 100” means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “treat,” “treated,” or “treating” mean both therapeutic treatments wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” means an activity that alleviates or ameliorates any of the primary phenomena or secondary symptoms associated with the cancer or any other condition described herein. In some embodiments, the cancer that is being treated is one of the cancers recited herein.

As used herein, the term “subject” can be used interchangeably with the term “patient”. The subject can be a mammal, such as a dog, cat, monkey, horse, or cow, for example. In some embodiments, the subject is a human. In some embodiments, the subject has been diagnosed with lung cancer. In some embodiments, the subject is believed to have lung cancer. In some embodiments, the subject is suspected of having lung cancer.

As used herein, the term “express” as it refers to a cell surface receptor, such as, but not limited to, CD3, CD4, and CD8, can also be referred to as the cell being positive for that marker. For example, a cell that expresses CD3 can also be referred to as CD3 positive (CD3+).

The term “cancer” as used herein is defined as disease characterized by the rapid and uncontrolled growth of aberrant cells. Cancer cells can spread locally or through the bloodstream and lymphatic system to other parts of the body. Cancer can occur in ‘liquid’ form, and appear in the blood, lymph, or other liquid forms, and can also occur in ‘solid’ form, such as a tumor, and appear in any organ in the body, including but not limited to lung, prostate, breast, brain, and the like.

“Enriched” as used herein is understood to mean a process so as to add or increase the proportion of a desirable ingredient or characteristic. Enrichment is understood as increasing the proportion of a specific cell type from a population of cells, e.g. peripheral blood or bone marrow, for the presence of a specific cell type, particularly immune cells based on the presence or absence of specific cell surface markers or metabolic characteristics. Enrichment includes cell sorting by methods such as flow cytometry which rely on sorting based on markers. Enriched, as used herein, does not include treating a cell population with a specific agent, such as an antibody or drug to increase the proliferation of one or more cell types and/or suppress the proliferation of one or more cell types.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein, “marrow infiltrating lymphocytes” or “MILs” are a subpopulation of immune cells and are described for example in, U.S. Pat. No. 9,687,510, which is hereby incorporated by reference in its entirety. MILs significantly differ from peripheral blood lymphocytes (PBLs). For example, MILs are more easily expanded, upregulate activation markers to a greater extent than PBLs, maintain more of a skewed Vβ repertoire, traffic to the bone marrow, and most importantly, possess significantly greater tumor specificity. In some embodiments, MILs can be activated, for example, by incubating them with anti-CD3/anti-CD-28 beads and under hypoxic conditions, as described herein. In some embodiments, growing MILs under hypoxic conditions is also described in U.S. Pat. No. 9,687,510, and International Application No. WO2016/037054, both of which are incorporated by reference herein in their entirety.

In some embodiments, methods to prepare MILs may include removing cells from the bone marrow, lymphocytes, and/or marrow infiltrating lymphocytes from the subject; incubating the cells in a hypoxic environment, thereby producing hypoxic-activated MILs. In some embodiments, the subject has cancer. The cells can also be activated in the presence of anti-CD3/anti-CD28 antibodies and cytokines as described herein.

Bone marrow may be collected from a patient having cancer that has been previously treated with a check point inhibitor. The checkpoint inhibitor can be an anti-PD-1 antibody, an anti-PD-L1 antibody, or a combination of these. The patient may have non-metastatic or metastatic disease at the time of the bone marrow removal. The patient may have been previously treated with chemotherapy or not.

The collected bone marrow may be frozen or immediately used, for example, to create tumor specific MILs. If the bone marrow is frozen, it is preferably thawed before incubation. The bone marrow may be treated to purify MILs through methods known to one of ordinary skill in the art. The MILs may be activated, for example, with beads, e.g., anti-CD4/CD28 beads. The ratio of beads to cells in the solution may vary; in some embodiments, the ratio is 3 to 1. Similarly, the MILs may be expanded in the presence of one or more antibodies, antigens, and/or cytokines, e.g., in the absence of anti-CD3/CD28 beads. The cell count for the collected bone marrow may be determined, for example, to adjust the amount of beads, antibodies, antigens, and/or cytokines to be added to the MILs. In some embodiments, MILs are captured using beads specifically designed to collect the cells.

The collected MILs can be grown in a hypoxic environment for a first period of time. The hypoxic environment may include less than about 7% oxygen, such as less than about 7%, 6%, 5%, 4%, 3%, 2%, or 1% oxygen. For example, the hypoxic environment may include about 0% oxygen to about 7% oxygen, 0% oxygen to about 6% oxygen, such as about 0% oxygen to about 5% oxygen, about 0% oxygen to about 4% oxygen, about 0% oxygen to about 3% oxygen, about 0% oxygen to about 2% oxygen, about 0% oxygen to about 1% oxygen. In some embodiments, the hypoxic environment includes about 1% to about 5% oxygen. In some embodiments, the hypoxic environment is about 1% to about 2% oxygen. In some embodiments, the hypoxic environment is about 0.5% to about 1.5% oxygen. In some embodiments, the hypoxic environment is about 0.5% to about 2% oxygen. The hypoxic environment may include about 7%, 6%, 5%, 4%, 3%, 2%, 1%, or about 0% oxygen, and all fractions thereof in between these amounts.

Incubating MILs in a hypoxic environment may include incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may include incubating the MILs for about 1 hour to about 30 days, such as about 1 day to about 20 days, about 1 day to about 14 days, or about 1 day to about 12 days. In some embodiments, incubating MILs in a hypoxic environment includes incubating the MILs in a hypoxic environment for about 2 days to about 5 days. The method may include incubating MILs in a hypoxic environment for about 1 day, 2 days, 3 days, 4 days, 5 days, 6 days, 7 days, 8 day, 9 days, 10 days, 11 days, 12 days, 13 days, or 14 days. In some embodiments, the method includes incubating the MILs in a hypoxic environment for about 3 days. In some embodiments, the method includes incubating the MILs in a hypoxic environment for about 2 days to about 4 days. In some embodiments, the method includes incubating the MILs in a hypoxic environment for about 3 days to about 4 days.

In some embodiments, hypoxic-activated MILs are then cultured in a normoxic environment to produce the therapeutically activated marrow infiltrating lymphocytes. In some embodiments, the normoxic environment may include at least about 7% oxygen. In some embodiments, the normoxic environment may include about, such as about 8% oxygen to about 30% oxygen, 10% oxygen to about 30% oxygen, about 15% oxygen to about 25% oxygen, about 18% oxygen to about 24% oxygen, about 19% oxygen to about 23% oxygen, or about 20% oxygen to about 22% oxygen. In some embodiments, the normoxic environment includes about 21% oxygen.

In some embodiments, the MILs are cultured in the presence of IL-2 or other cytokines. In some embodiments, the MILs are cultured in normoxic conditions in the presence of IL-2. In some embodiments, the other cytokines can be IL-7, IL-15, IL-9, IL-21, or any combination thereof. In some embodiments, the MILs can be cultured in cell culture medium that includes one or more cytokines, e.g., such as IL-2, IL-7, and/or IL-15, or any suitable combination thereof. Illustrative examples of suitable concentrations of each cytokine or the total concentration of cytokines includes, but is not limited to, about 25 IU/mL, about 50 IU/mL, about 75 IU/mL, about 100 IU/mL, about 125 IU/mL, about 150 IU/mL, about 175 IU/mL, about 200 IU/mL, about 250 IU/mL, about 300 IU/mL, about 350 IU/mL, about 400 IU/mL, about 450 IU/mL, or about 500 IU/mL or any intervening amount. In some embodiments, the cells are cultured in about 100 IU/mL of each of, or in total of, IL-2, IL-1, and/or IL-15, or any combination thereof. In some embodiments, the cell culture medium includes about 250 IU/mL of each of, or in total of, IL-2, IL-1, and/or IL-15, or any combination thereof.

Incubating MILs in a normoxic environment may include incubating the MILs, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 1 1 days, 12 days, 13 days, or even at least about 14 days. Incubating may include incubating the MILs for about 1 hour to about 30 days, such as about 1 day to about 20 days, about 1 day to about 14 days, about 1 day to about 12 days, or about 2 days to about 12 days.

In some embodiments, the MILs are obtained by extracting a bone marrow sample from a subject and culturing/incubating the cells as described herein. In some embodiments, the bone marrow sample is centrifuged to remove red blood cells. In some embodiments, the bone marrow sample is not subject to apheresis. In some embodiments, the bone marrow sample does not include PBLs or the bone marrow sample is substantially free of PBLs. These methods select for cells that are not the same as what have become to be known as tumor infiltrating lymphocytes (“TILs”), which have distinct limitations for use in adoptive T cell therapy. Thus, a MIL is not a TIL. In some embodiments, the bone marrow sample contains less than 10%, 9%, 8%, 7%, 6%, 5%, 4%, 3%, 2%, or less than 1% PBLs as compared to the total of MILs. In some embodiments, the sample is free of PBLs.

In some embodiments, the cells are also activated by culturing with antibodies to CD3 and CD28. This can be performed, for example by incubating the cells with anti-CD3/anti-CD28 beads that are commercially available or that can be made by one of skill in the art. The cells can then be plated in a plate, flask, or bag. Hypoxic conditions can be achieved by flushing either the hypoxic chamber or cell culture bag for 3 minutes with a 95% Nitrogen and 5% CO₂ gas mixture. This can lead to, for example, 1-2% or less 02 gas in the receptacle. Examples of such beads and methods of stimulation can be found, for example, in U.S. Pat. Nos. 6,352,694, 6,534,055, 6,692,964, 6,797,514, 6,867,041, and 6,905,874, each of which is incorporated by reference in its entirety. Alternatives to beads include engineered cells, such as K562 cells, that can be used to stimulate the MILs. Such methods can be found in, for example, U.S. Pat. Nos. 8,637,307 and 7,638,325, each of which is incorporated by reference in its entirety. Cells can also be stimulated using other methods, such as those described in U.S. Pat. No. 8,383,099, which is incorporated by reference in its entirety.

In multiple myeloma (MM), bone marrow infiltrating lymphocytes (MILs) have shown increased anti-tumor reactivity and proliferative capacity compared to their peripheral blood counterparts. The CD8⁺ T cell compartment in peripheral blood and bone marrow (BM) is diverse and includes several subsets with different phenotype, function, metabolic requirements and gene expression profiles.

In some embodiments, the MILs are enriched for or selected for certain metabolic characteristics. Enriching the MILs is an important step for successful adoptive T cell therapy, as the efficacy can be significantly increased when treating various forms of cancer. Naïve and memory T cells are metabolically quiescent, favoring oxidative phosphorylation (OXPHOS) over glycolysis. In contrast, effector T cells favor glycolysis to fuel their rapid proliferation. The role that fatty acid (FA) uptake and oxidation (FAO) plays in the maintenance of certain T-cell phenotypes has been recently illuminated, specifically tissue resident memory T-cells (T_{RM})—a subtype that appears to be increasingly important in generating and maintaining the tumor specificity of MILs. It is becoming clearer that hypoxia plays an important role in enhancing the efficacy of MILs in adoptive T cell therapy, especially when compared to that of peripheral blood lymphocytes (PBLs).

Specifically, unique metabolic and gene expression profiles are described herein for hypoxia-activated MILs, further elucidating the unique phenotype of the MILs, which show greater overall expansion and enhanced tumor-specificity. This unique metabolic profile shows upregulation of both oxidative phosphorylation (OXPHOS) and the glycolytic machinery, suggesting that hypoxia-activated MILs possess properties of both effector and memory cells, which likely accounts for the observed enhanced anti-tumor activity. In contrast, PBLs grown under the same conditions fail to expand significantly and lack the metabolic differences seen in MILs.

The hypoxia-activated MILs further demonstrate upregulation of several genes involved in fatty acid uptake and oxidation including leptin, ghrelin, FABP5, and CD36, as shown by RNAseq. RT-PCR shows greater expression of anti-apoptotic proteins in the hypoxia-activated MILs, including Bcl-2, BclxL, VHL, and NOS. Increased expression of cytokines can also be observed in the hypoxia-activated MILs, including ILp-2, IL-7m IL-15, IFNγ, and IL-2Ra. The post-expansion MILs, using intracellular staining and FACs analysis, show an increase in mitochondrial proteins, including TOMM20, CPT1a, and SDHa, increased mTOR signaling, as well as increases in the glycolytic machinery—HK2 and GLUT1. A higher baseline oxygen consumption rate, spare respiratory capacity, and extracellular acidification rate in the hypoxia-activated MILs is also observed via the Seahorse metabolic flux analysis.

In one embodiment, hypoxia-activated MILs are enriched for their unique metabolic profile, resulting in a tissue resident memory T-cells (T_{RM}) population which can be used to enhance efficacy of adoptive T cell therapy. Hypoxia-activated MILs are shown to be long-lasting, persistent, and possess anti-tumor properties, and thus can be effective in ACT.

In addition to a unique metabolic profile, the hypoxia-activated MILs also display a unique immunophenotype that can also be used to further enrich the MILs to obtain a more efficacious population of T cells. Specifically, described herein are populations of CD8⁺ T cells that are used to identify immunophenotypes that are exclusively present within the bone marrow. specifically, several CD8⁺ putative subpopulations and their frequencies in each sample type are identified using a combination of tSNE for dimensionality reduction and Phenograph for unsupervised clustering (see Mair et al., Eur J. of Immunol. “The end of gating? An introduction to automated analysis of high dimensional cytometry data”, (2016) 46:34-43).

In one embodiment, two subpopulations of CD8⁺ T cell that are uniquely present in the bone marrow and virtually absent in peripheral blood were identified as expressing CD69. Definition of the phenotypic identity of these two clusters revealed a shared signature characterized by a CD69⁺ PD1^{int/hi} CD57⁻ effector-like phenotype and lack of additional activation and exhaustion markers. These CD8⁺ T cell subsets were absent in the peripheral blood compartment, suggesting that these subpopulations are tumor specific. Both clusters of CD69⁺ CD8⁺ MILs expressed increased levels of CXCR6 and CXCR4, thereby partially sharing the core signature of tissue-resident memory T cells. Interestingly, CD127 and CD27/CD28 expression highlighted the dichotomy between the phenotypes of these two BM resident CD8⁺ T cell subsets. Specifically, CD127⁺ CD69⁺CD8⁺ MILs expressed higher levels of TCF1, indicating increased proliferative capacity and stemness, lower levels of TOX, suggesting partial exhaustion with retained effector potential and intermediate levels of exhaustion markers such as TIGIT and PD1. In contrast, CD127⁻ CD69⁺ CD8⁺ MILs displayed a more exhausted/dysfunctional phenotype, while retaining partial effector function as suggested by very low CD57 expression levels.

As described herein, CD127⁺ CD69⁺ CD8⁺ MILs may be responsible for maintenance of long-term disease control and may be linked to increased patient survival. Enriching for these cells can be critical for successful adoptive T cell therapy, especially in the post-transplant setting where activated MILs therapy has already been proven feasible.

In some embodiments, the composition includes a population of tumor cancer specific MILs that are CD3 positive. In some embodiments, at least about, or at least, 40% of the MILs are CD3 positive. In some embodiments, about, or at least, 45%, 50%, 55%, 60%, 65%, 70%, 75%, 80%, 85%, 86%, 87%, 88%, or 89% of MILs are CD3 positive. In some embodiments, at least, or about, 80% of the MILs are CD3 positive. In some embodiments, about 40% to about 100% of the MILs are CD3 positive. In some embodiments, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 86% to about 100%, about 87% to about 100%, about 88% to about 100%, or about 90% to about 100% of the MILs are CD3 positive (express CD3).

In some embodiments, the composition includes either a population of MILs that do not express CD3, or a population of MILs that expresses low levels of CD3, for example, relative to the expression level of MILs from the population of MILs that express CD3.

In some embodiments, the composition includes a population of MILs that expresses interferon gamma (“IFNγ”), i.e., wherein each cell in the population of MILs that expresses IFNγ is a marrow infiltrating lymphocyte that expresses IFNγ, e.g., as detected by flow cytometry. For example, at least about 2% of the cells in the composition may be MILs that express IFNγ, or at least about 2%, 3%, 4%, 5%, 6%, 7%, 8%, 9%, 10%, 11%, 12%, 13%, 14%, 15%, 16%, 17%, or even at least about 18% of the MILs express IFNγ. In some embodiments, about 2% to about 100% of the MILs express IFNγ, such as about 2% to about 100%, about 3% to about 100%, about 4% to about 100%, about 5% to about 100%, about 6% to about 100%, about 7% to about 100%, about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, or even about 18% to about 100% of the MILs. In some embodiments, the composition includes either a population of MILs that do not express IFNγ, e.g., as detected by flow cytometry, or a population of MILs that expresses low levels of IFNγ, i.e., relative to the expression level of MILs from the population of MILs that express IFNγ.

In some embodiments, the composition includes a population of MILs that expresses CXCR4. For example, at least about 98% of the MILs express CXCR4, such as at least about 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, or even at least about 99.7% of the MILs. In some embodiments, about 98% to about 100% may be MILs that express CXCR4, such as at least about 98.1% to about 100%, about 98.2% to about 100%, about 98.3% to about 100%, about 98.4% to about 100%, about 98.5% to about 100%, about 98.6% to about 100%, about 98.7% to about 100%, about 98.8% to about 100%, about 98.9% to about 100%, about 99.0% to about 100%, about 99.1% to about 100%, about 99.2% to about 100%, about 99.3% to about 100%, about 99.4% to about 100%, about 99.5% to about 100%, about 99.6% to about 100%, or even about 99.7% to about 100% of the MILs in the composition. In some embodiments, the composition includes either a population of MILs that do not express CXCR4, e.g., as detected by flow cytometry, or a population of MILs that expresses low levels of CXCR4, i.e., relative to the expression level of MILs from the population of MILs that express CXCR4.

In some embodiments, the composition includes a population of MILs that expresses CD4. The population of MILs that expresses CD4 may include a plurality of MILs that expresses CXCR4.

The population of MILs that expresses CD4 may include a plurality of MILs that expresses 4-1BB. For example, at least about 21% of the cells in the composition may be MILs from the plurality of MILs that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to about 100% of the cells in the composition may be MILs from the plurality of MILs that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition.

The composition may include a population of MILs that expresses CD8. The population of MILs that expresses CD8 may include a plurality of MILs that expresses CXCR4.

The population of MILs that expresses CD8 may include a plurality of MILs that expresses 4-1BB. For example, at least about 21% of the cells in the composition may be MILs from the plurality of MILs that expresses 4-1BB, such as at least about 8%, 9%, 10%, 11%, 12%, 13%, 14%, 15%, 16%, 17%, 18%, 19%, 20% or even at least about 21% of the cells in the composition. In some embodiments, about 2% to about 100% of the cells in the composition may be MILs from the plurality of MILs that expresses 4-1BB, such as about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, about 18% to about 100%, about 19% to about 100%, about 20% to about 100%, or even about 21% to about 100% of the cells in the composition.

In some embodiments, the composition includes a population of MILs that expresses 4-1BB. For example, at least about 21% of the cells in the composition may be MILs from the population of MILs that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to 100% of the cells in the composition may be MILs from the population of MILs that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition. In some embodiments, the composition includes either a population of MILs that do not express 4-1BB, e.g., as detected by flow cytometry, or a population of MILs that expresses low levels of 4-1BB, i.e., relative to the expression level of MILs from the population of MILs that express 4-1BB.

In some embodiments, the composition includes MILs that express CD4. In some embodiments, the composition includes MILs that express CD8. In some embodiments, the ratio of CD4⁺:CD8⁺ MILs present in the composition is about 2:1.

The composition may include a population of MILs that expresses CD8. The population of MILs that expresses CD8 may include a plurality of MILs that expresses CXCR4.

In some embodiments, the composition includes a population of MILs that expresses CD4. The population of MILs that expresses CD4 may include a plurality of MILs that expresses CXCR4.

The MILs may express the different factors or surface receptors as described herein alone or in combination with one another. Thus, for example, a MIL can be CD3+, CD4+, and/or CD8+. Such cells can also express IFNγ. The cells can also be positive or negative for the various factors or receptors provided for herein.

In some embodiments, activated MILs and/or therapeutic activated MILs are administered to a subject having, or suspected of having, cancer. In some embodiments, hypoxic-activated MILs and/or therapeutic activated MILs are produced from a bone marrow sample from a subject having or suspected of having cancer, then administering to the same subject to treat cancer. In some embodiments, the MILs are allogeneic to the subject.

In some embodiments, the MILs can be administered in a pharmaceutical preparation or pharmaceutical composition. Pharmaceutical compositions including the tumor cancer specific MILs may further include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated for parenteral administration, e.g., intravascular (intravenous or intraarterial), intraperitoneal or intramuscular administration. In some embodiments, the MILs and/or compositions are administered by parenteral administration, e.g., intravascular (intravenous or intraarterial), intraperitoneal or intramuscular administration. The compositions can also be administered directly into the tumor. In some embodiments, the compositions are administered intravenously.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the subject can be pre-conditioned with cyclophosphamide with or without fludarabine. One such example is provided for in U.S. Pat. No. 9,855,298, which is hereby incorporated by reference. Another non-limiting example is administering fludarabine (30 mg/m² intravenous daily for 3 days) and cyclophosphamide (300 mg/m² intravenous daily for 3 days starting with the first dose of fludarabine). After administration, the MILs™ can be administered after, e.g., 2 to 14 days after, completion of the fludarabine and cyclophosphamide. In some embodiments, the cyclophosphamide is administered or 2-3 days at a dose of about 300 to about 600 mg/m².

In some embodiments, the pharmaceutical composition that is administered includes tumor cancer-specific MILs as provided for herein. A composition of such MILs is also provided for herein. In some embodiments, the tumor cancer specific MILs are hypoxic activated. In some embodiments, the tumor cancer-specific MILs are hypoxic activated/normoxic activated MILs. A tumor cancer-specific MIL is a MIL that can specifically target cancer in a subject.

### EXAMPLES

### Example 1: Activation of Marrow Infiltrating Lymphocytes in Hypoxia Generates T-Cells with Enhanced Anti-Tumor Activity and a Unique Profile, Resembling that of Tissue-Resident Memory Cells

Activating MILs in hypoxia generates T cells with a greater overall expansion compared to PBLs, a unique subset post-transplant of stem-like CD8+ T cells with the bone marrow only in patients receiving hypoxia-activated MILs (aMILs), and, most strikingly, a metabolic profile that upregulates both OXPHOS and glycolytic machinery. This metabolic profile, seen only when the MILs are activated under hypoxic conditions, suggests that hypoxia aMILs possess properties of both effector and memory cells and are more stem-like—features that may account for the observed enhancements in anti-tumor activity, memory, and persistence. In contrast, PBLs grown under the same conditions fail to expand significantly, show limited tumor specificity, and lack the metabolic differences seen in MILs.

RNAseq revealed that hypoxia aMILs upregulate several genes involved in FAO, as well as genes associated with stemness, cellular adhesion, and activation. Using intracellular staining and FACS analysis, the postexpansion MILs product was found to possess increased mitochondrial proteins, increased mTOR signaling, and increases in glycolytic machinery. Seahorse metabolic flux analysis revealed a higher baseline oxygen consumption rate and extracellular acidification rate in the hypoxia-activated MILs and a trend towards increased spare respiratory capacity. Further flow analysis identified a unique CD69+ CD127+ population found in the BM of patients receiving an infusion of hypoxia aMILs. Taken together, these data suggest that by activating MILs in hypoxia, we are enriching for a hybrid TSCM/TRM-like population. Such enrichment could have significant implications in enhancing the efficacy of ACT.

### Materials and Methods

Bone marrow and blood are collected from consenting myeloma patients. Samples are processed and lymphocytes isolated by Ficoll density gradient centrifugation and frozen for later use. For cell activation and Tcell enrichment, samples are slow-thawed and plated at 2×106 cells/mL in a U-bottom 96 well plate in AIM V media supplemented with IL-2. Anti-CD3/CD28 dynabeads are added for activation. Cells are cultured initially in hypoxia, then removed for IL-2 supplementation, further expansion, and downstream experiments.

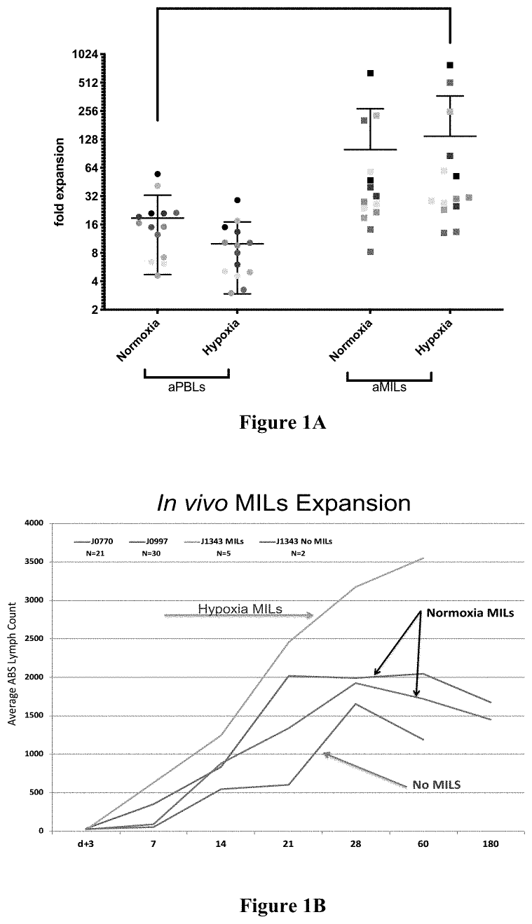

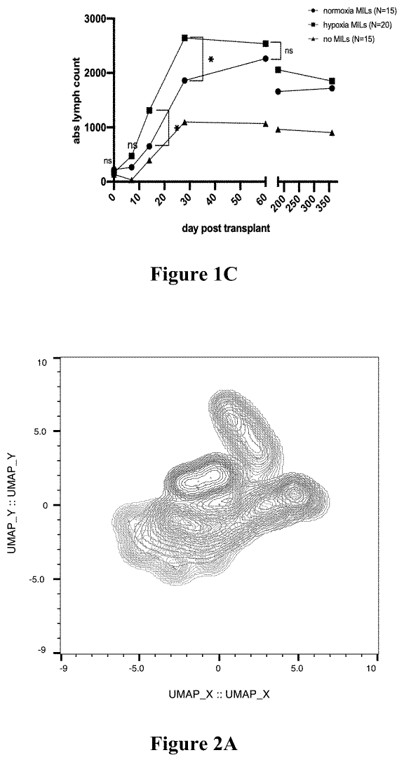

As shown in FIG. 1A-1C, MILs expand more significantly than PBLs and this proliferative advantage is maintained when activated in hypoxia. When MILs are used clinically, hypoxia-activated MILs also expand more in vivo, as measured by absolute lymph count (see 1B and 1C).

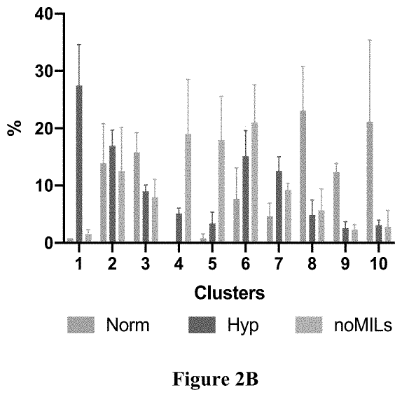

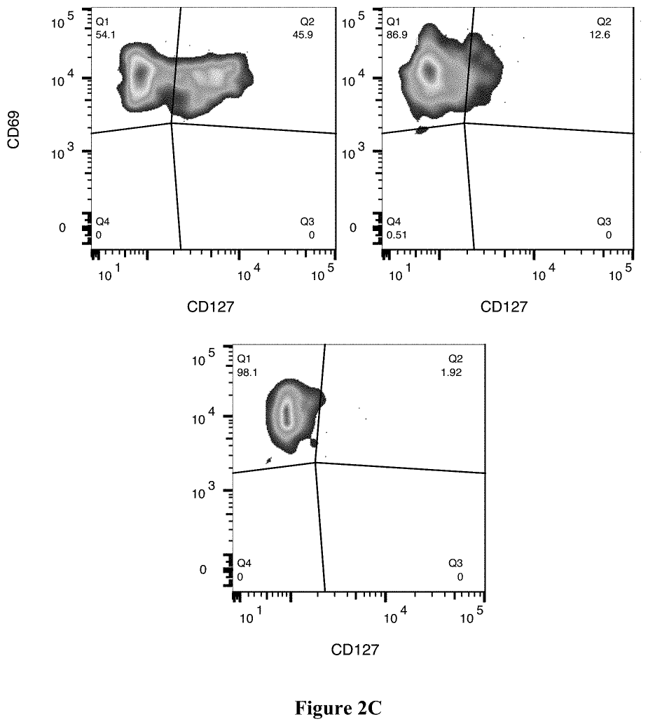

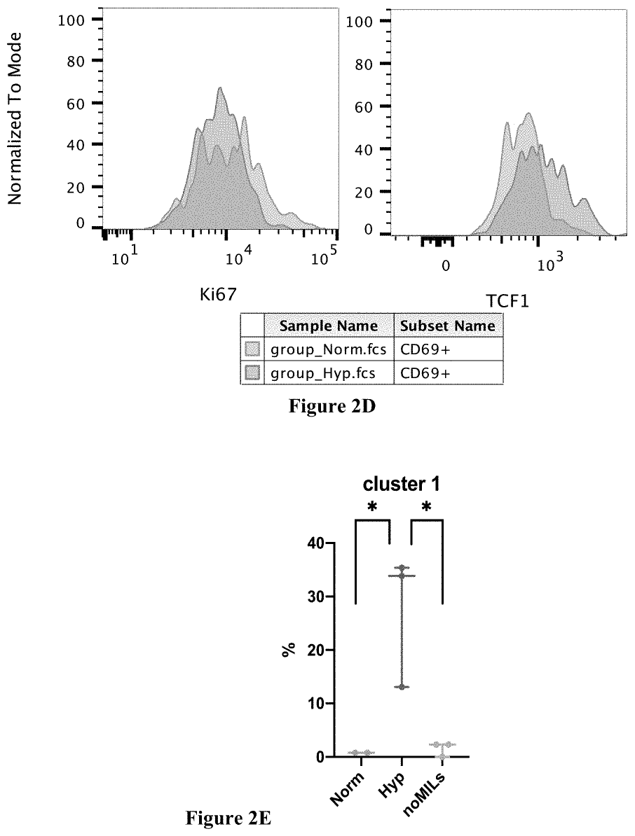

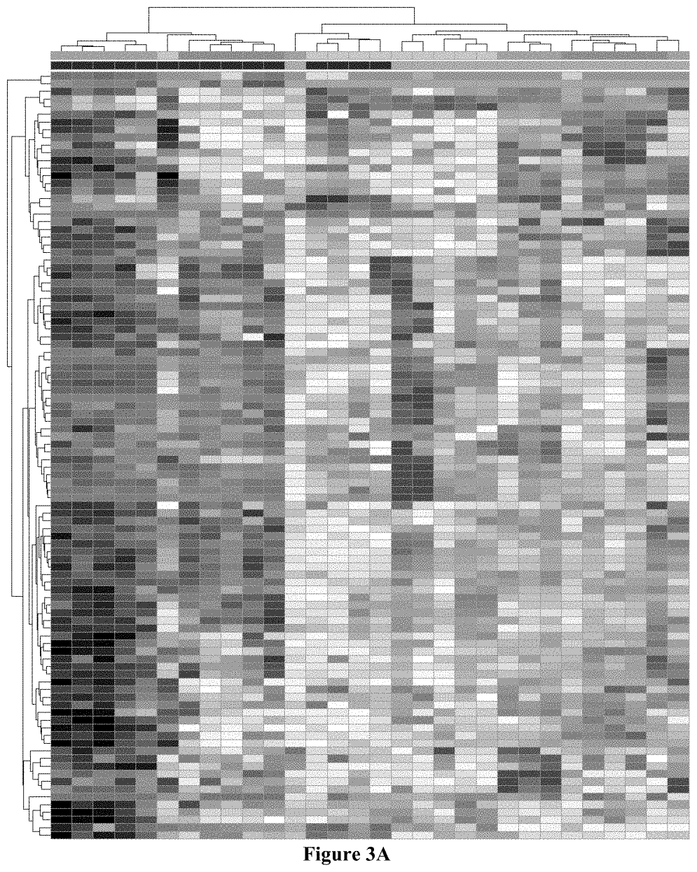

FIG. 2A-2E shows that a unique population of CD8+ CD69+ CD127+ cells was identified in the bone marrow only of patients receiving hypoxic aMILs and not normoxic aMILs or traditional transplant without aMILs. FIG. 2A-2B shows use of the uniform manifold approximation and projection (UMAP) data analysis. The CD69+ cells within the hypoxia aMILs group are also Ki67 low and TCF1 high, suggesting that they are hypo-proliferative and stemlike, while in normoxia, the inverse is true.

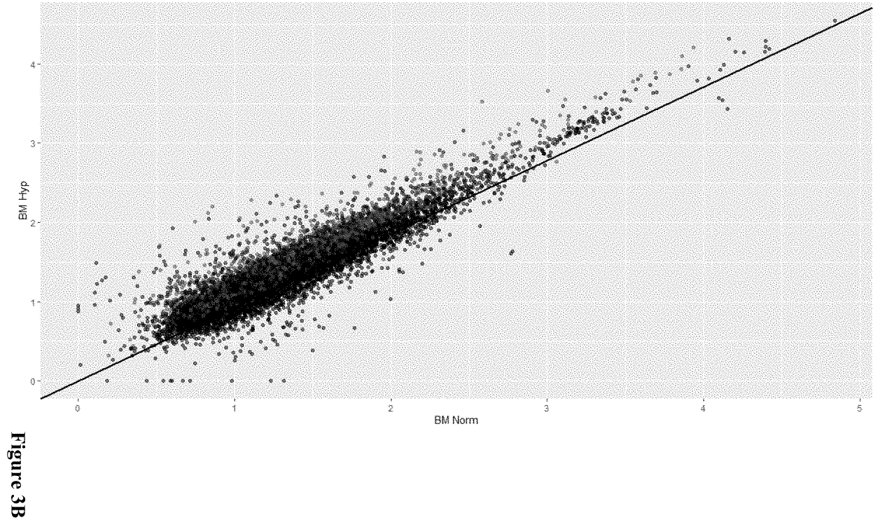

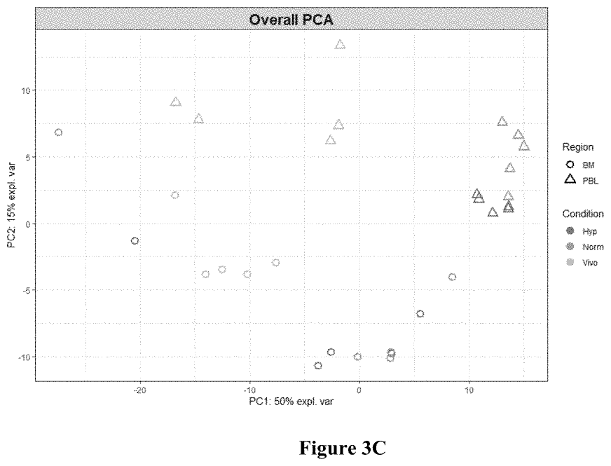

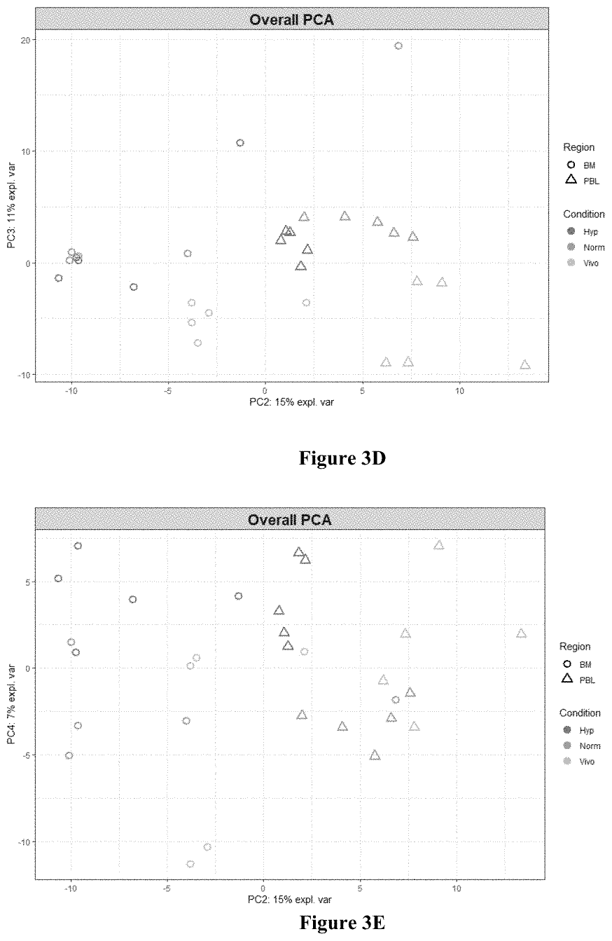

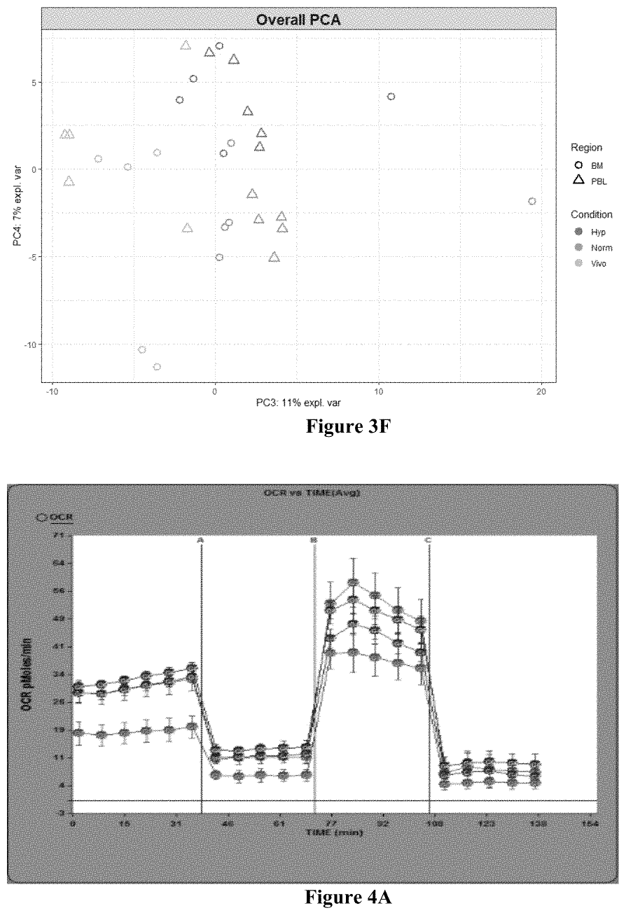

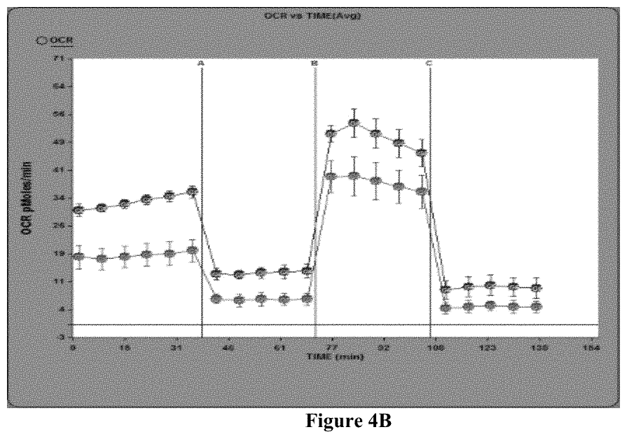

FIG. 3 shows the results of RNA-sequencing, revealing unique gene expression patterns in aMILs following initial hypoxic activation. Five paired samples (matched bloods and bone marrows from the same patients) were processed and activated in either normoxic or hypoxic conditions. Sequencing was run on isolated CD3+ T-cells. Genes of interest include KLF4, APOE, CD36, Rab32, and IL18 as well as many others involved in metabolism, activation, adhesion, and stemness.

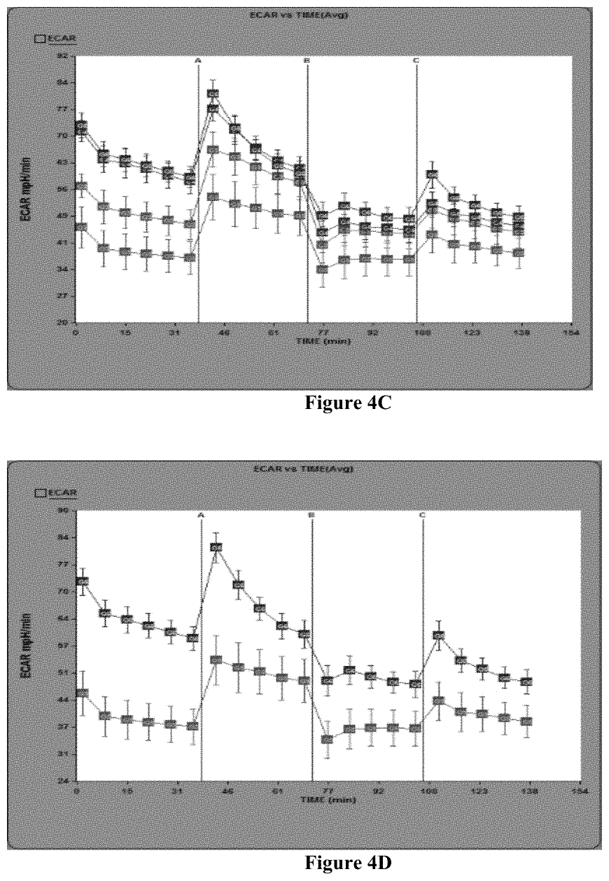

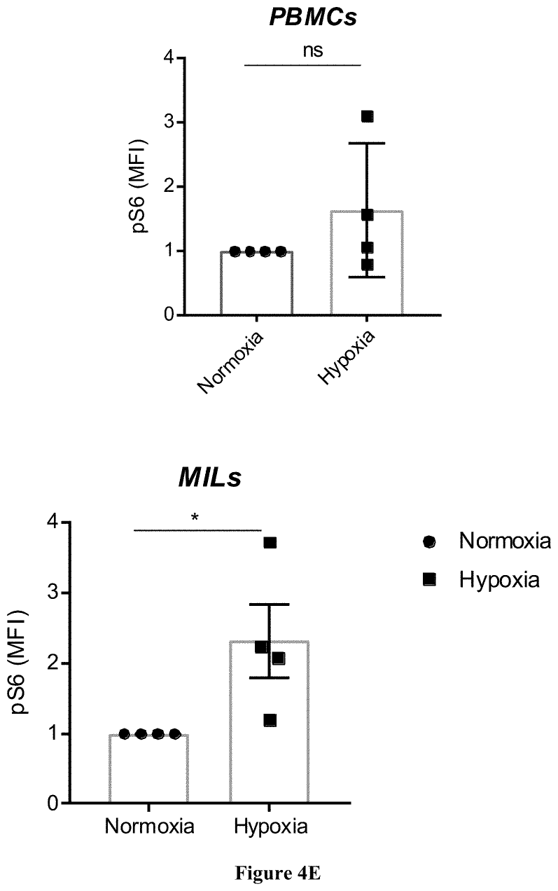

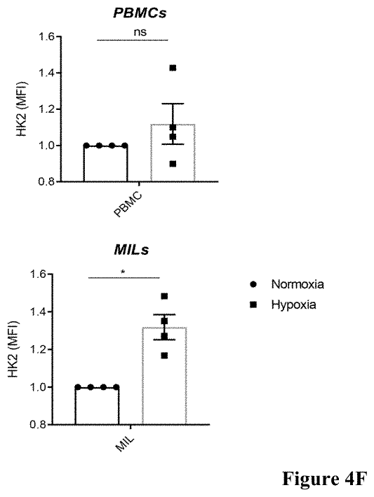

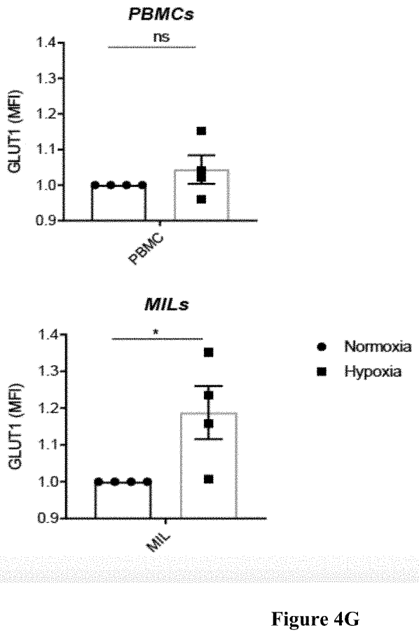

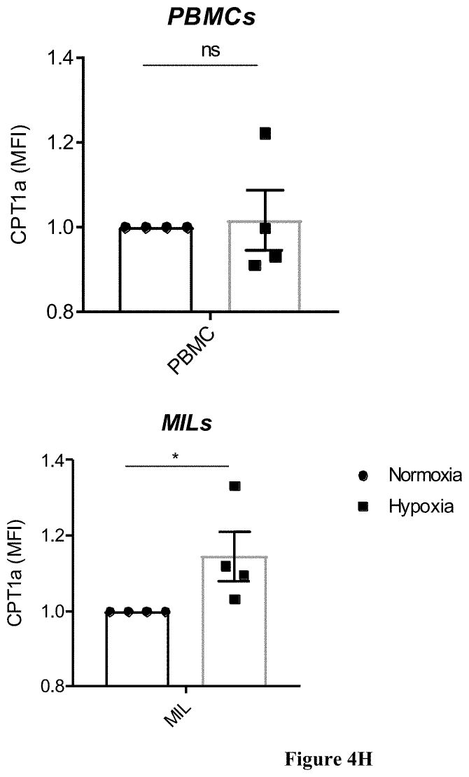

FIG. 4A-41 shows the results of Seahorse analysis measuring oxygen consumption rate (OCR) and extracellular acidification rate (ECAR) of hypoxic aMILs, showing that oxidative respiration and glycolysis are upregulated in hypoxic aMILs. They have a higher baseline O₂ consumption and increased expression of mitochondrial proteins as well as increased mTOR signaling. Glycolysis and glycolytic machinery are also upregulated in MILs activated in hypoxia. They have a higher ECAR and increased uptake of glucose when compared to both normoxic aMILs and hypoxic aPBLs. Seahorse traces are representative of 5 independent experiments.

The bone marrow has long been thought to serve as a reservoir for long-lived memory T-cells with stem-like abilities to remain quiescent until needed to mount an immune attack. This data shows that MILs are less proliferative and more stem-like than PBLs at baseline but, upon activation in hypoxia, they rapidly begin to proliferate while retaining memory for specific antigens. Like long lived memory cells, hypoxia aMILs have upregulated OXPHOS and mitochondrial function but, like effector cells, hypoxia aMILs also have increased glycolytic machinery and ECAR. Further analysis by RNAseq has identified many of the genes involved in these processes as uniquely upregulated by MILs in hypoxia.

### Example 2: High-Dimensional Analysis of Marrow-Infiltrating Lymphocytes Identifies Stem-Like, Bone Marrow-Resident CD8+ T Cells with Increased Tumor Specificity in Multiple Myeloma

High dimensional analysis of CD8 T cells from individuals with newly diagnosed multiple myeloma (NDMM) was conducted, and subsets were identified that are enriched in the bone marrow compared to peripheral blood. Besides exhausted and senescent cells, BM-enriched T cells were identified with a partial exhausted phenotype that retained markers of stem-like plasticity and proliferation capacity. In addition, we defined a peculiar BM niche that may form a supportive microenvironment for BM-resident T cells. Hence, this data shows that MILs may be responsible for maintenance of long-term disease control and may be linked to increased patient survival.

### Materials and Methods

Sample collection and processing: BM and PB samples were collected from patients with multiple myeloma. All patients provided written informed consent. After density separation of mononuclear cells, samples were either stained for flow cytometry or cryopreserved until further use.

Multicolor flow cytometry: Frozen peripheral blood (PB) and bone marrow (BM) samples were thawed and, after washing in PBS, stained with a combination of fluorochrome conjugated monoclonal antibodies and a live/dead cell marker. Intranuclear staining was performed following fixation of cells and by incubating with specific antibodies. Sample data were acquired on a 10-color Gallios® flow cytometer.

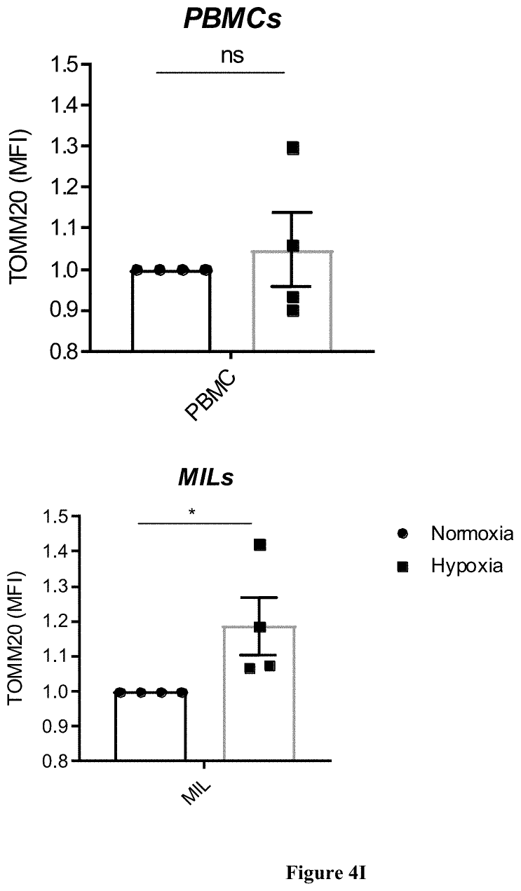

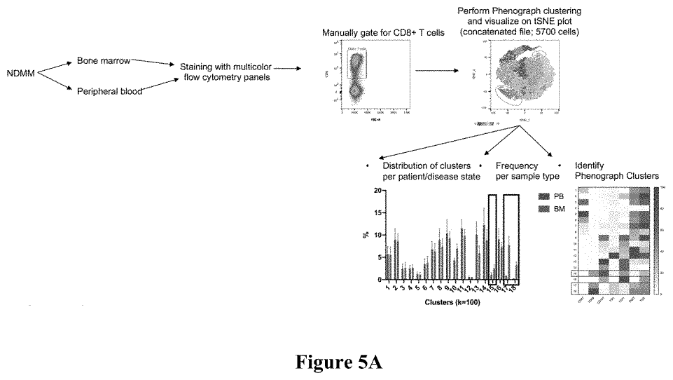

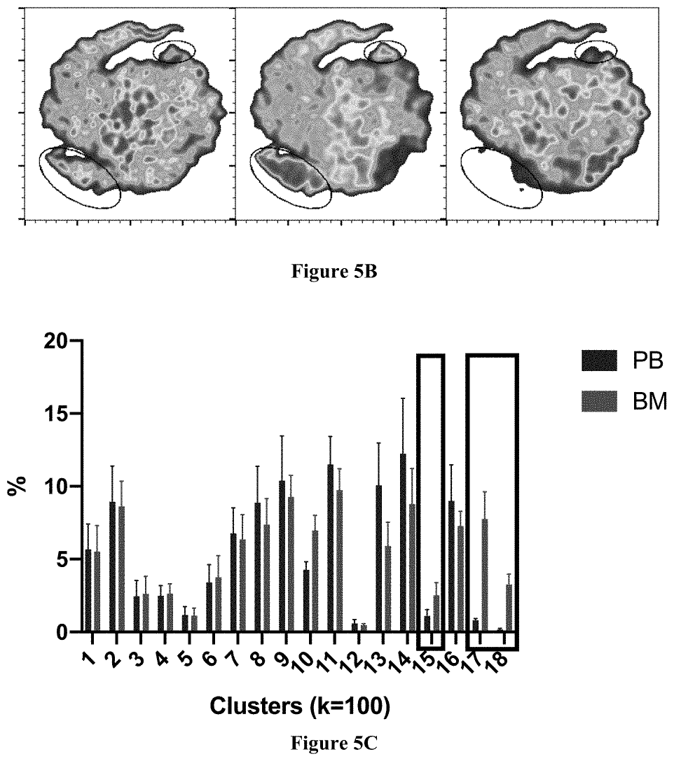

Flow cytometry data analysis and statistical analysis: After manual gating for CD8+ T cells, combined use of tSNE for dimensionality reduction and Phenograph for unsupervised clustering allowed for the characterization of T cell subsets. See the experimental workflow in FIG. 5A. Statistical analyses were performed using GraphPad Prism®, Rstudio® and FlowJo® analysis software. A p value of less than 0.05 was considered to be significant.

FIG. 5B-5C show the results of high-dimensional analysis of CD8+ T cells, and identifies BM enriched subsets. tSNE analysis of concatenated CD8+ T cells from PB and BM of NDMM patients (top panel, n=7). Frequency of the 18 Phenograph clusters in PB (blue) and BM (red) samples (bottom panel).

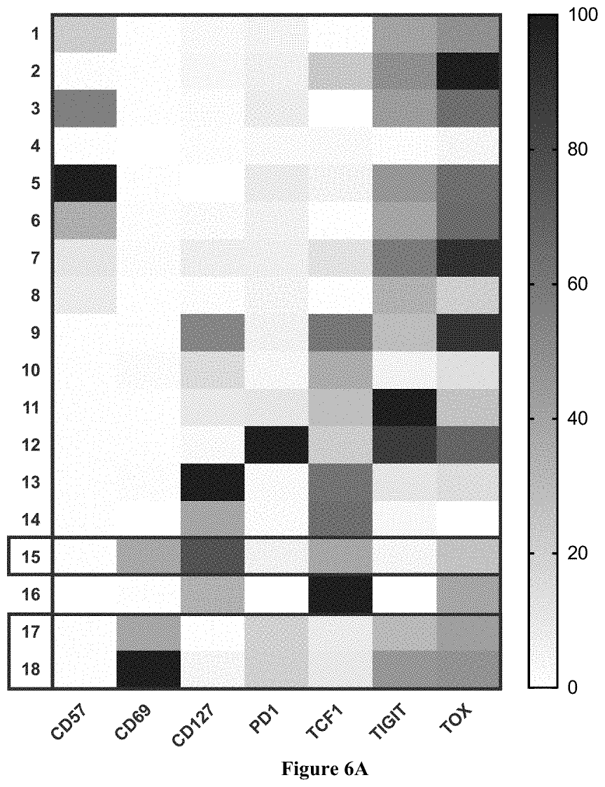

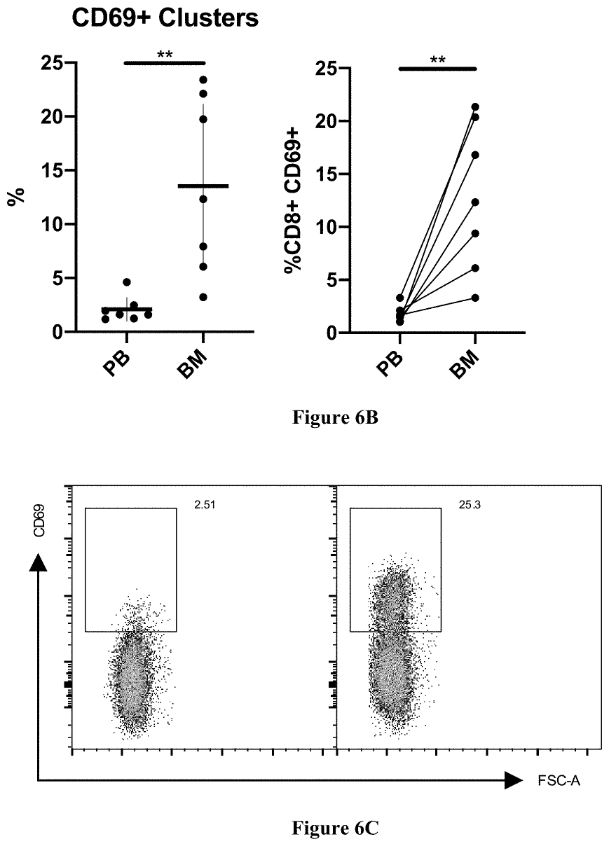

The data in FIGS. 6A-6C show that multiple myeloma BM T cells are enriched in CD69+ CD8+ T cells. FIG. 6A shows a heatmap showing the integrated median fluorescence intensity (MFI) of specific markers in discrete Phenograph clusters identified in FIG. 5C. Clusters 15, 17 and 18 were significantly enriched in the BM (see FIG. 6B, left panel). The median frequencies of clusters 15, 17 and 18 along with CD69+ CD8+ T cells within PB and BM samples are depicted in FIG. 6B top right panel (**, p<0.01). Representative dot plots showing manual gating of with CD69+ CD8+ population in PB (left) and BM (right) samples (FIG. 6C bottom right panel).

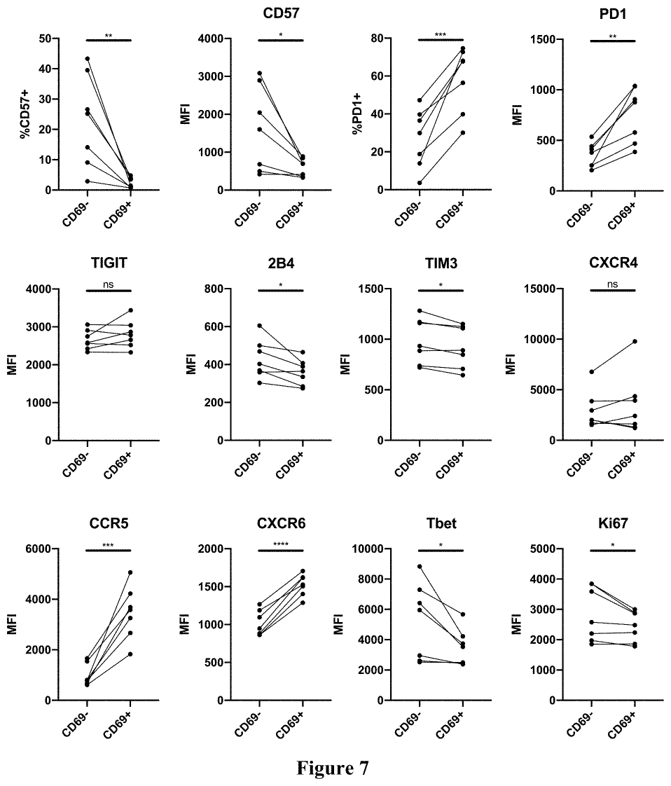

The data in FIG. 7 show that BM CD69+ CD8+ T cells display an effector phenotype and a tissue resident-like (TRM) signature. BM TRM cells display a partial exhausted phenotype as exemplified by expression of PD1 and lack of TIM3, TIGIT and 2B4. Higher expression of CXCR6 and CCR5 associated with lower Tbet and Ki67 levels suggests retained stemlike/proliferative capacity.

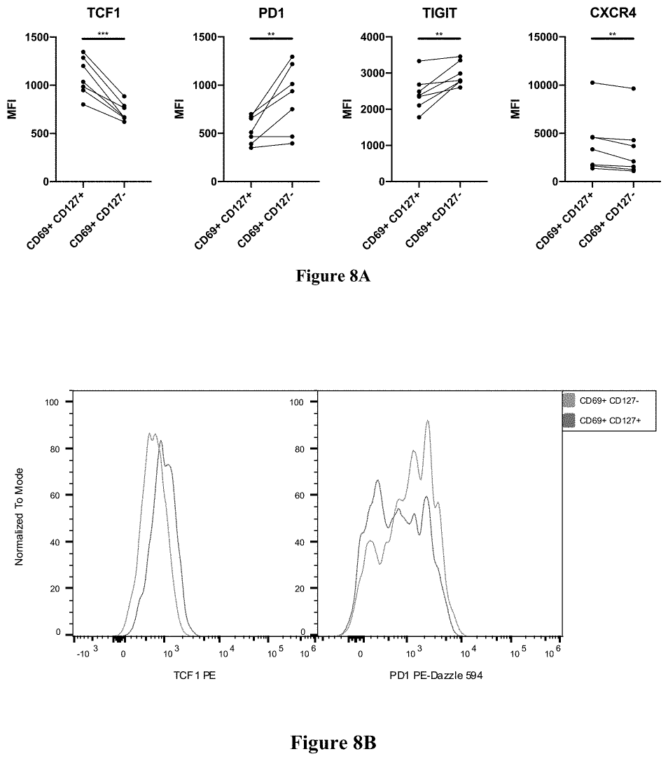

The data in FIG. 8A shows that CD127 (known as an IL7 receptor) identifies a subset of stem-like BM-resident CD8+ T cells. Phenograph clustering identified Cluster 15 as BM-enriched and CD127+, while both Cluster 17 and 18 lack CD127 expression. CD69+CD127+ CD8+ T cells display increased expression of the stemness marker TCF1, as well as lower PD1 and TIGIT levels (FIG. 8A). Representative histograms depicting expression of TCF1 and PD1 in CD69+ CD127+ and CD69+ CD127− (FIG. 8B)

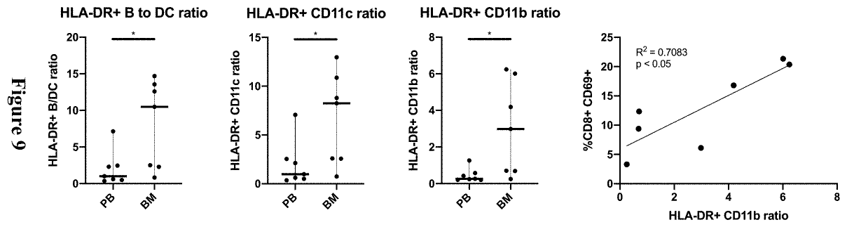

The data in FIG. 9 show that HLA-DR+ APCs in the BM may form a supportive niche for BM-enriched CD8+ T cells via the identification of APC subsets in PB and BM samples. BM APCs are characterized by a higher frequency of B cells and lower frequencies of both CD11c+ and CD11b+ HLA-DR+ APCs (bottom/left three panels). The ratio of CD11b−/CD11b+ HLA-DR+ APCs positively correlates with the frequency of CD8+ CD69+ T cells in the BM suggesting the existence of a BM-restricted niche that may support and differentiate stem-like CD8+ T cells thereby maintaining an effective anti-myeloma immune response (top/right panel).

Unsupervised clustering with Phenograph identifies CD69+ CD8+ T cells as a subset of cells restricted to the BM and virtually absent in PB. Despite displaying a partial exhausted phenotype, CD69+ CD8+ T cells lack 2B4, TIM3 and CD57 expression, suggesting retained proliferative capacity and stem-like features. Among CD69+ CD8+ T cells, CD127 expression identifies a stem-like subset that could potentially be a precursor of more exhausted and terminally differentiated CD8+ T cells. The data shows that stem-like MILs are a crucial component for successful adoptive cell therapy approaches in multiple myeloma, hematologic malignancies, and solid tumors. A BM niche of antigen presenting cells may maintain and differentiate stem-like cytotoxic T cells.

Therefore, high-dimensional single cell analysis of CD8+ T cells of NDMM patients has identified BM-enriched immunophenotypes characterized by baseline CD69 expression, and CD69+ CD127+ T cells represent a distinct population of BM-resident T cells that displays a stem-like phenotype, and lack of CD11b expression on BM APCs positively correlates with CD69+ BM T cells.

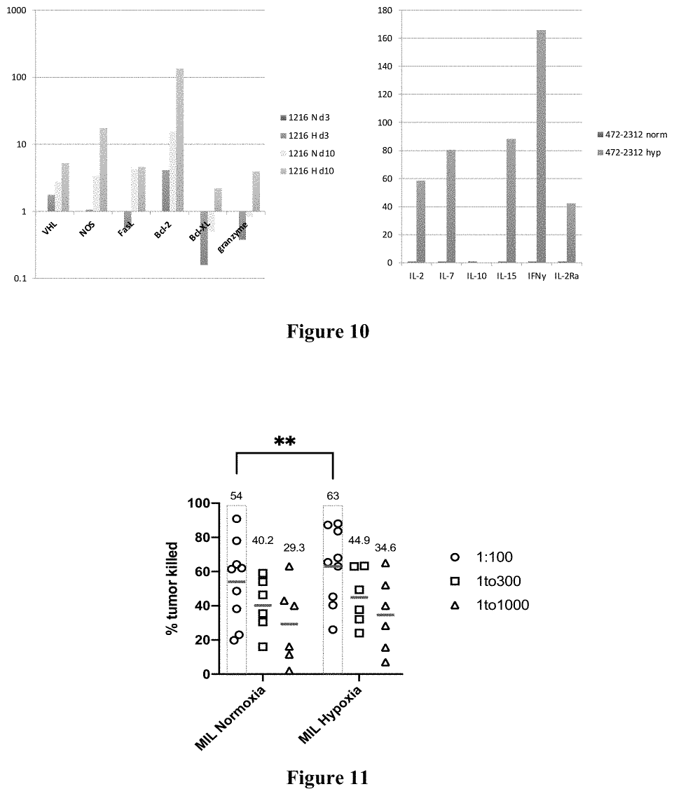

The data in FIG. 10 shows the results of experiments wherein MILs were either grown in normoxia or hypoxia for 3 days and then converted to normoxia for a total of 10 days in culture. RT-PCR was performed for anti-apoptotic proteins of the cells either immediately after coming out of hypoxia or 7 days later. Increased expression of Bcl-2, Bcl_{XL} as well as the hypoxia-inducible genes, VHL and NOS was observed that was sustained at the late time point. This also correlated with upregulation of a favorable cytokine profile as shown by greater expression of IL-2, IL-7, IL-15, IFNγ and IL-2Ra. Taken together, this data suggests that hypoxia generates favorable changes within the cell which prevents their death and enhances their survival.

FIG. 11 shows a Seahorse analysis showing a distinctive pattern of hypoxic MILs. Specifically, they had an increase in baseline 02 consumption, spare respiratory capacity, mitochondrial mass, mTOR signaling. This all translated into better tumor-specific cytotoxicity of autologous myeloma cells. It should be noted that in general an effector:target (E:T) ratio of 1:100 is already quite low which intrinsically speaks to the enhanced properties of MILs. The fact that hypoxia further augmented this effect further supports the hypothesis that hypoxia induces significant beneficial changes to MILs.

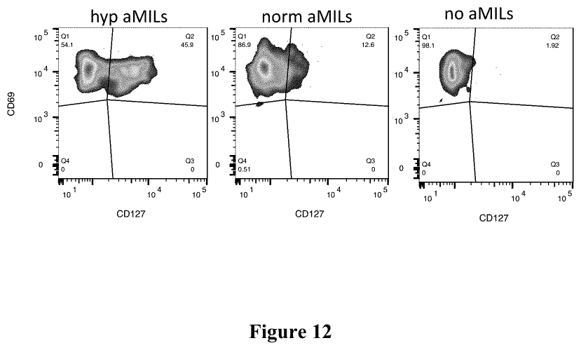

FIG. 12 demonstrates that CD69+ tissue resident memory (Trm) play a significant role in mediating the local tissue protective immunity and immunosurveillance. They are enriched in the BM and play a key role in imparting long-term immunity. FIG. 12 shows flow cytometry using BM from patients undergoing a stem cell transplant transplanted either without MILs or MILs grown in normoxia or hypoxia and obtained on day 28 post-transplant. Expansion in hypoxia upregulates CD69 expression on MILs with co-expression of the IL7R (CD127)—a marker of long-term memory T cells.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method of enriching for tumor-specific MILs, comprising:
I) preparing MILs from the bone marrow of a cancer patient;
II) evaluating the MILs for gene expression, metabolic profile, or phenotype;
III) selecting and isolating the MILs that exhibit the gene expression, metabolic profile, or phenotype;
wherein said gene is selected from the group consisting of: IL-2, IL-7, IL-15, IFNγ. IL-2Ra, CD69, CXCR6, CXCR4, CD127, TCF1, leptin, ghrelin, FABP5, CD36, and combinations thereof;
wherein said metabolic profile is selected from the group consisting of upregulation of oxidative phosphorylation, upregulation of glycolytic machinery, fatty acid oxidation, and combinations thereof; and
wherein said phenotype is selected from the group consisting of:
a) an increase in mitochondrial proteins selected from the group consisting of TOMM20, CPT1a, SDHa, and combinations thereof,
b) an increase in mTOR signaling,
c) an increase in glycolytic machinery selected from the group consisting HK2, GLUT1, and combinations thereof,
d) increased baseline oxygen consumption rate,
e) spare respiratory capacity,
f) extracellular acidification rate, and
g) combinations thereof.

2. The method of claim 1, wherein said preparing comprises expanding the MILs under hypoxic conditions.

3. The method of claim 2, wherein said hypoxic conditions comprise incubating the MILs in an environment having about 0% oxygen to about 6% oxygen.

4. The method of claim 2, wherein said hypoxic conditions comprise incubating the MILs in an environment having less than 5% oxygen.

5. The method of claim 2, wherein said hypoxic conditions comprise incubating the MILs in an environment having between 0.5% and 1.5% oxygen.

6. The method of claim 1, wherein said preparing expanding the MILs under hypoxic conditions for about 1 to 12 days.

7. The method of claim 1, wherein said preparing expanding the MILs under hypoxic conditions for about 2 to 5 days.

8. The method of claim 1, wherein said preparing expanding the MILs under hypoxic conditions for about 3 to 4 days.

9. The method of claim 1, wherein said preparing comprises expanding the MILs under hypoxic conditions, followed by culturing the hypoxic-activated MILs in a normoxic environment.

10. The method of claim 9, wherein said normoxic environment comprises at least about 7% oxygen.

11. The method of claim 9, wherein said normoxic environment comprises about 7% to about 21% oxygen.

12. The method of claim 9, wherein said normoxic environment comprises about 21% oxygen.

13. A composition comprising tumor-specific MILs produced by the method of claim 1.

14. The composition of claim 13, wherein the tumor-specific MILs comprise characteristics as compared to PBLs or T cells grown under normoxic-only conditions, wherein the characteristics are selected from the group consisting of:
1) increased cytotoxicity,
2) persistence over time in vivo,
3) inducement of long-term memory,
4) expression of a beneficial cytokine profile for cytotoxicity, and
5) combinations thereof.

15. A method of treating a subject having cancer, said method comprising administering the composition of claim 2 to the subject.

16. The method of claim 15, wherein the cancer is a hematological cancer.

17. The method of claim 16, wherein the hematological cancer is multiple myeloma.

18. The method of claim 15, wherein the cancer is a solid tumor.

19. A method of isolating tumor-specific MILs comprising incubating bone marrow aspirate from a patient having cancer in a hypoxic environment, followed by culturing in a normoxic environment, wherein the tumor specific MILs comprise characteristics as compared to PBLs or T cells grown under normoxic-only conditions, wherein the characteristics are selected from the group consisting of:
1) increased cytotoxicity,
2) persistence over time in vivo,
3) inducement of long-term memory,
4) expression of a beneficial cytokine profile for cytotoxicity, and
5) combinations thereof.
